(19) United States
(12) Patent Application Publication
(10) Pub. No.: US 2014/0005450 A1
(43) Pub. Date: Jan. 2, 2014

(54) USE OF N-PARAFFIN ADSORPTION TO INCREASE SELECTIVITY AND YIELD OF SYNTHETIC DISTILLATE FUEL

(71) Applicants: Terry L. Marker, Palos Heights, IL (US); John A. Petri, Wauconda, IL (US); Charles P. Luebke, Mt. Prospect, IL (US); Tom N. Kalnes, LaGrange, IL (US); Michael J. McCall, Geneva, IL (US)

(72) Inventors: Terry L. Marker, Palos Heights, IL (US); John A. Petri, Wauconda, IL (US); Charles P. Luebke, Mt. Prospect, IL (US); Tom N. Kalnes, LaGrange, IL (US); Michael J. McCall, Geneva, IL (US)

(73) Assignee: UOP LLC, Des Plaines, IL (US)

(21) Appl. No.: 13/537,535

(22) Filed: Jun. 29, 2012

(51) Int. Cl. C07C 5/13

(52) U.S. Cl. 585/310

(57) ABSTRACT

Methods of making synthetic distillate fuel are described. The methods involve the use of an absorbent bed of molecular sieves which adsorb the n-paraffins from a distillate fuel cut. This allows the distillate fuel true boiling point cut point on the distillation column to increase to a higher temperature to make a distillate fuel which meets all of the synthetic paraffinic kerosene (SPK) or synthetic diesel specifications on distillation as well as the cold flow property specification, such as freeze point for SPK or cloud point, cold filter plugging point and pour point for synthetic diesel. This approach could improve aviation fuel yields by about 5 to about 10% and synthetic diesel yields up to 20%.

### FIELD OF THE INVENTION

This invention relates to a process for producing synthetic distillate fuel boiling range hydrocarbons useful as aviation fuel and diesel fuel from renewable feedstocks such as the glycerides and free fatty acids found in materials such as plant oils, animal oils, animal fats, and greases, and other renewable and non-renewable sources of normal paraffins. The process involves the use of n-paraffin adsorption beds to improve the yield and selectivity of the synthetic distillate fuel.

### BACKGROUND OF THE INVENTION

As the demand for fuels such as aviation and diesel fuel increases worldwide, there is increasing interest in sources other than petroleum crude oil for producing the fuels. One source is renewable feedstocks including, but not limited to, plant oils such as corn, jatropha, camelina, rapeseed, canola, soybean and algal oils, animal fats such as tallow, fish oils, and various waste streams such as yellow and brown greases and sewage sludge. The common feature of these feedstocks is that they are composed of mono- di- and tri-glycerides, and free fatty acids (FFA). Another class of compounds appropriate for these processes is fatty acid alkyl esters (FAAE), such as fatty acid methyl ester (FAME) or fatty acid ethyl ester (FAEE). These types of compounds contain aliphatic carbon chains generally having from about 8 to about 24 carbon atoms. The aliphatic carbon chains in the glycerides, FFAs, or FAAEs can be saturated or mono-, di- or poly-unsaturated. Most of the glycerides in the renewable feed stocks will be triglycerides, but some may be monoglycerides or diglycerides. The monoglycerides and diglycerides can be processed along with the triglycerides. Such renewable feedstocks after hydrotreating in a hydrotreating zone under hydrotreating conditions will provide a mixture of almost entirely n-paraffins.

Another non-petroleum source for the production of aviation and diesel fuel boiling range hydrocarbons is liquids produced from Fischer-Tropsch synthesis. Non-biorenewable feedstocks such as, but not limited to, natural gas, shale gas, coal, petroleum coke, and poly-olefins such as polyethylene and polypropylene, are transformed into synthesis gas by steam reforming or gasification processes well known in the art. Biorenewable sources such as, but not limited to, municipal solid wastes or ligno-cellulosic based components such as wood, switchgrass, begasse, tall oil and others are also transformed into synthesis gas by steam reforming or gasification processes. Synthesis gas is a mixture of carbon monoxide, carbon dioxide, hydrogen and water and may also contain impurities that are detrimental to the Fischer-Tropsch synthesis process. These impurities are removed, and the hydrogen to carbon monoxide ratio is adjusted to the proper ratio required for the Fischer-Tropsch synthesis. The liquids from such a synthesis process can contain primarily normal paraffins, linear olefins and linear alcohols with the carbon numbers of these liquids typically varying from four to over one hundred. The linear olefins and linear alcohols are typically prominent in the lighter hydrocarbons that boil in the diesel boiling range or lighter, for example, in carbon numbers less than twenty five. The liquids from a Fischer-Tropsch synthesis process can be used to make renewable aviation or diesel fuel when the feed source for the steam reforming or gasification is a biorenewable source. The liquids from a Fischer-Tropsch synthesis process can be used to make a non-biorenewable aviation or diesel fuel when the feed source for the steam reforming or gasification is not from a biorenewable source. Fischer-Tropsch synthesis processes can be broadly categorized into high-temperature Fischer-Tropsch and low-temperature Fischer-Tropsch synthesis processes. The ratio between the linear and branched components in the Fischer-Tropsch liquids is discussed in “Fischer-Tropsch Fuels Refinery Design”, Energy Environ. Sci., 2011, 4, 1177. In a high-temperature synthesis process, the ratio between linear and branched molecules typically ranges from 2:1 to 4:1, and the normal paraffin content after a hydrotreatment step would typically be 66 to 80 mass percent. In a low-temperature synthesis process, the ratio between linear and branched molecules is typically higher than 20:1, and the normal paraffin content after a hydrotreatment step would typically be greater than 95 mass percent. Overall, Fischer-Tropsch liquids after hydrotreating in a hydrotreating zone under hydrotreating conditions will provide a mixture of substantially n-paraffins with the normal paraffins comprising about 50 to 100 mass percent of the mixture.

There are references disclosing the production of hydrocarbons from oils. For example, U.S. Pat. No. 4,300,009 discloses the use of crystalline aluminosilicate zeolites to convert plant oils (e.g., corn oil) to hydrocarbons (e.g., gasoline), and chemicals (e.g., para-xylene). U.S. Pat. No. 4,992,605 discloses the production of hydrocarbon products in the diesel boiling range by hydroprocessing vegetable oils such as canola or sunflower oil. Finally, US 2004/0230085 A1 discloses a process for treating a hydrocarbon component of biological origin by hydrodeoxygenation followed by isomerization.

### SUMMARY OF THE INVENTION

One aspect of the present invention relates to a method of making synthetic distillate fuel. Synthetic distillate fuel refers to aviation fuel, jet fuel, kerosene, synthetic paraffinic kerosene (SPK), No. 2 fuel oil, diesel fuel, gas oil and like fuels that meets fuel specifications discussed below or similar fuel specifications and is not derived from petroleum-based feedstocks.

In one embodiment, the method includes hydrotreating a feedstock in a hydrotreating zone under hydrotreating conditions to obtain a mixture of substantially n-paraffins. At least a portion of the n-paraffins are isomerized and hydrocracked in an isomerization and hydrocracking zone under mild isomerization and hydrocracking conditions to obtain a mixture of n-paraffins and isomerized paraffins. At least a portion of the mixture of n-paraffins and isomerized paraffins are separated in a bed of molecular sieves into an n-paraffin stream consisting essentially of n-paraffins and an isomerized paraffin stream consisting essentially of isomerized paraffins. The isomerized paraffin stream is fractionated into at least a heavy distillate fraction consisting essentially of isomerized paraffins, and a light distillate fraction consisting essentially of isomerized paraffins.

In another embodiment, the feedstock is hydrotreated in a hydrotreating zone under hydrotreating conditions to obtain a mixture of substantially n-paraffins. At least a portion of the n-paraffins are isomerized and hydrocracked in an isomerization and hydrocracking zone under mild isomerization and hydrocracking conditions to obtain a mixture of n-paraffins and isomerized paraffins. The mixture of n-paraffins and isomerized paraffins are fractionated into at least a heavy distillate fraction, and a light distillate fraction. The heavy distillate fraction is separated in a bed of molecular sieves into a heavy distillate n-paraffin stream consisting essentially of n-paraffins and a heavy distillate isomerized paraffin stream consisting essentially of isomerized paraffins.

### BRIEF DESCRIPTION OF THE DRAWING

FIG. 1 illustrates one embodiment of a process for making aviation and diesel fuel from renewable feedstocks.

FIG. 2 is a general flow schematic of one embodiment of a process utilizing the present invention.

FIG. 3 is a general flow schematic of another embodiment of a process utilizing the present invention.

### DETAILED DESCRIPTION OF THE INVENTION

Synthetic distillate fuel can be made by hydroprocessing various feedstocks to produce aviation and diesel range hydrocarbons. The hydrotreating stage produces a mixture of substantially n-paraffins. By substantially n-paraffins, we mean that at least about 50% of the net products produced in the hydrotreating stage are n-paraffins.

The process also produces paraffinic green diesel, paraffinic green naphtha, and liquified petroleum gas (LPG), which is substantially propane, normal butane and isobutane. In a second stage, the n-paraffins are isomerized and mildly cracked to improve the cold properties of the resulting paraffins.

Any feedstock that produces substantially n-paraffins during hydroprocessing can be used. Suitable feedstocks include, but are not limited to biorenewable feedstocks containing glycerides or free fatty acids, other feedstocks derived from biorenewable sources, such as Fischer-Tropsch liquids obtained from the gasification or steam reforming of biorenewable feed sources, and feedstocks derived from non-biorenewable sources, such as Fischer-Tropsch liquids obtained from the gasification or steam reforming of a non-biorenewable feed source.

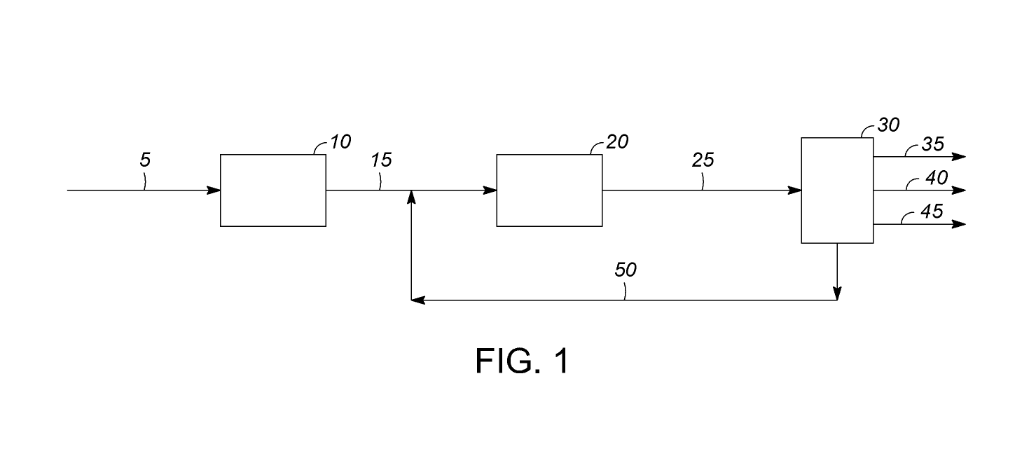

FIG. 1 illustrates one such process. The feedstock 5 is sent to a hydroprocessing zone 10 where is it reacted with hydrogen to form n-paraffins 15. The n-paraffins typically range from C5 to C30, depending on the feedstock used. The n-paraffin effluent 15 enters isomerization and hydrocracking zone 20 where the n-paraffins are isomerized and hydrocracked. The effluent 25 from the isomerization and hydrocracking zone 20 is sent to a fractionator 30, where it is separated into one or more streams. Typical streams include C1-C4 light ends 35, C5-C8 light naphtha and heavy naphtha 40, and distillate 45. A recycle stream 50 is recycled to the isomerization and hydrocracking zone 20.

The distillate 45 can be diesel fuel or aviation fuel that meets the diesel or aviation fuel specifications. For example, diesel fuel has to substantially meet flash point, ASTM D-86 or D-2887 distillation specifications, and a cold flow property, such as cloud point. Aviation fuel has to substantially meet flash point, ASTM D-86 or D-2887 distillation specifications and a cold flow property such as freeze point. To make arctic diesel or aviation fuel, the True Boiling Point (TBP) cut point between the distillate 45 and the recycle 50 is typically about 254° C. (490° F.) to about 266° C. (510° F.) in order to meet distillation and cold flow properties simultaneously. The recycle 50 is iso-paraffins and n-paraffins that boil with a TBP cut higher than the recycle oil cut point.

The distillation curves of the liquid streams 40, 45 and 50 produced from fractionator 30 are typically measured with standardized ASTM methods, such as ASTM D86 and ASTM D2887. These typical laboratory distillation measurements from ASTM D86 or D2887 are converted to true boiling point distillation curves for each stream 40, 45 and 50 using standard distillation inter-conversion methods well known in the art. In another example, the true boiling point distillations of the liquid streams 40, 45 and 50 from fractionator 30 can be directly measured using ASTM D2892 or ASTM D5236. The true boiling point distillations from these liquid streams are mathematically blended together based on the proportion of volume or weight in which they are produced to generate an overall TBP curve of the total liquid effluent from fractionator 30. The TBP cut point between the distillate 45 and recycle oil 50 is determined from the weight or volume fraction of the recycle oil relative to the total liquid effluent from fractionator 30. A non-limiting example follows. For example, if the recycle oil 50 comprises fifty two liquid volume percent of the total liquid effluent from fractionator 30, then the temperature at which one hundred minus fifty two liquid volume percent of the total liquid effluent boils is the TBP cut point between the recycle oil 50 and the next lightest cut in fractionator 30, which is distillate stream 45.

One difficulty with this process is that, in some embodiments such as the embodiment shown in FIG. 1, a TBP cut point of about 254° C. (490° F.) to about 266° C. (510° F.) has to be used when separating the final aviation fuel fraction from the diesel fuel fraction in order to obtain the desired freeze point specification in the aviation fuel. Freeze point of an aviation fuel is measured typically with a standardized method such as ASTM D2386. ASTM D1655 requires Jet A aviation fuel to meet a maximum freeze point of −40° C. or Jet A-1 aviation fuel to meet a maximum freeze point of −47° C. These ASTM D1655 specifications are for aviation fuel derived from petroleum crude oil sources. ASTM D7566 requires SPK aviation fuel to meet a maximum freeze point of −40° C. However, producers of aviation fuels may require more stringent specifications for the SPK freeze point when SPK is blended with aviation fuel produced from petroleum crude oil sources as a finished product for use in aviation services. This TBP cut point is lower than would be required for the distillation specifications, such as distillation end point. A typical distillation specification for both ASTM D1655 and ASTM D7566 is 300° C. maximum final boiling point using ASTM D86. The reason for the difference is the presence of heavier n-paraffins which control the freeze point characteristics of the aviation fuel. K. Petrovic and D. Vitorovic (J. of the Institute of Petroleum, Vol. 59 (565), p. 20-26) concluded that a linear relationship existed between experimentally determined freeze points and the total content of the heaviest three carbon numbers of the n-paraffins in the aviation fuel sample tested. The TBP cut point of about 254° C. (490° F.) to about 266° C. (510° F.) is required in the isomerization and hydrocracking zone 20 to isomerize and crack the heavier normal paraffins into substantially isomerized and lighter paraffins that meet the freeze point requirement of the aviation fuel. The heaviest normal paraffins for this TBP cut point are substantially C₁₄ and C₁₅ normal paraffins. The hydrocracking of these C₁₄ and C₁₅ normal paraffins leads to the significant production of light and heavy naphtha and less selective production of isomerized paraffins in the aviation fuel range.

The problem of low selectivity for the production of aviation and diesel fuel can be solved by adding an absorbent bed of molecular sieves which adsorb the n-paraffins from the aviation fuel cut or diesel fuel cut or both aviation and diesel fuel cuts, which can be referred to as synthetic distillate. For example, this will allow the TBP cut point on the distillation column between the aviation fuel and the recycle oil to increase to a higher temperature, e.g., about 316° C. (600° F.), to make an aviation fuel which meets all of the SPK specifications on distillation, as well as the freeze point specification. This approach improves aviation fuel yields by about 5 to about 10% by not having to recycle material with true boiling points between about 260° C. (500° F.) (e.g., about 254° C. (490° F.) to about 266° C. (510° F.)) and about 316° C. (600° F.).

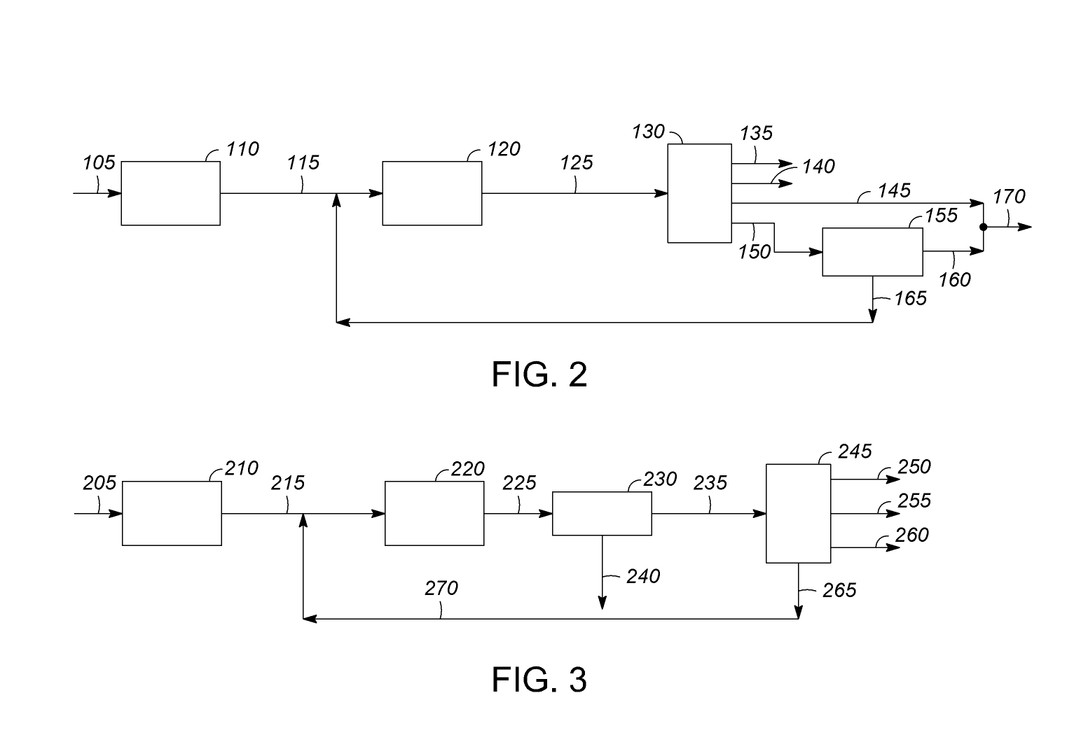

FIG. 2 illustrates one embodiment of the process of the present invention. The feedstock 105 is sent to the hydroprocessing zone 110 where is it reacted with hydrogen to form substantially n-paraffins 115. The n-paraffins typically range from C5 to C30, depending on the feedstock used. The n-paraffin effluent 115 enters isomerization and hydrocracking zone 120 where the n-paraffins are isomerized and hydrocracked under mild conditions.

The mild conditions are designed to maximize isomerization and minimize hydrocracking. Suitable mild conditions include a temperature in a range of about 260 to about 345° C. (500 to 650° F.), a pressure of about 1750 kPa(g) (about 250 psig) to about 6900 kPa(g) (about 1000 psig), a ratio of H₂:HC of about 1000 to about 5000 standard cubic feet per barrel (SCFB), and a liquid space hourly velocity (LHSV) of about 0.1 to about 5.

The mild conditions can be further defined based on the isomer to n-paraffin ratio of the products in the synthetic distillate carbon number range and extent of hydrocracking of the feedstock from the isomerization and hydrocracking zone. Lower isomer to n-paraffin ratios indicate less severe conditions in the isomerization and hydrocracking zone. Less severe conditions in the isomerization and hydrocracking zone also produce less undesirable products, such as light ends, LPG and light and heavy naphtha. A typical isomer to n-paraffin ratio from the isomerization and hydrocracking zone is less than 7:1, or less than 5:1, or less than 3:1.

One measure of the extent of hydrocracking of the feedstock in the isomerization and hydrocracking zone is the fraction of the feedstock that is converted into the undesirable products. Under the mild conditions of the present invention, about 0 to 25 mass percent of the feedstock to the isomerization and hydrocracking zone is converted to light ends, LPG, and light and heavy naphtha.

The effluent 125 from the isomerization and hydrocracking zone 120 is sent to a fractionator 130, where it is separated into one or more streams. Typical streams include C1-C4 light ends 135, C5-C8 light naphtha and heavy naphtha 140, light distillate 145 and heavy distillate 150. The light distillate 145 meets the cold flow property requirement of the light distillate. In one example, the light distillate 145 meets the freeze point specification of an aviation fuel. In another example, the distillate meets diesel fuel cold flow property specifications such as cloud point (ASTM D2500), cold filter plugging point (ASTM D6371), low temperature flow test (ASTM D4539), or any combination of these cold flow properties.

The heavy distillate 150, which contains iso-paraffins and n-paraffins, is sent to the bed of molecular sieves 155 where the iso-paraffins 160 are separated from the n-paraffins 165. The n-paraffins 165 are recycled back to the isomerization and hydrocracking zone 120. The iso-paraffins 160 meet the distillation specification, as well as the cold flow property requirement for arctic diesel cloud point or SPK freeze point. In another embodiment, the iso-paraffins meet the distillation specification, as well as the cold flow property requirements for diesel cold flow properties, which are less stringent than arctic diesel. The iso-paraffins 160 are combined with the light distillate 145 to form the diesel or aviation fuel 170. The distillation specification for aviation fuel would allow a TBP cut point between the aviation fuel 170 and the recycled oil 165 up to about 293° C. (560° F.) to about 316° C. (600° F.).

FIG. 3 shows another embodiment of the process of the present invention. The feedstock 205 is sent to the hydroprocessing zone 210 where is it reacted with hydrogen to form substantially n-paraffins 215. The n-paraffins typically range from C5 to C30, depending on the feedstock used. The n-paraffin effluent 215 enters isomerization and hydrocracking zone 220 where the n-paraffins are isomerized and hydrocracked under mild conditions, as discussed above.

All or a portion of the effluent 225 from the isomerization and hydrocracking zone 220 is sent to the bed of molecular sieves 230 where the iso-paraffins 235 are separated from the n-paraffins 240. The n-paraffins 240 are recycled back to the isomerization and hydrocracking zone 220.

The iso-paraffins 235 are sent to the fractionator 245, where they are separated into one or more streams. Typical streams include C1-C4 light ends 250, C5-C8 light naphtha and heavy naphtha 255, SPK or synthetic diesel 260, and heavy distillate 265.

Although the heavy distillate 265 is almost entirely iso-paraffins, it may not meet the distillation specification for the distillate, and it is recycled to the isomerization and hydrocracking zone 220. The distillation specification for aviation fuel would allow a TBP cut point between the SPK 260 and heavy distillate 265, which is the recycle oil, up to about 293° C. (560° F.) to about 316° C. (600° F.). The distillation specification for synthetic diesel would allow a TBP cut point between the synthetic diesel 260 and the heavy distillate 265 up to about 660° F. (350° C.) to about 720° F. (380° C.). The TBP cut point between the synthetic diesel 260 and the recycle oil 265 is based on meeting the ASTM D86 T90% maximum temperature specification for ASTM D975 or meeting the ASTM D86 T95% maximum temperature specification for European Union diesel fuel specifications.

The recycle oil 270 includes the n-paraffins 240 from the bed of molecular sieves 230 and the heavy distillate 265.

The severity of the isomerization and hydrocracking catalyst system is much lower because the TBP cut point between the SPK or synthetic diesel and the recycle oil has been increased about 28° C. (50° F.) to about 55° C. (100° F.).

The yield of aviation fuel could be nominally increased about 5 wt % to about 10 wt % based on meeting the aviation fuel distillation specification. The yield of synthetic diesel fuel could be nominally increased about 20 wt % based on meeting the diesel fuel distillation specification. The aviation fuel yield increase and synthetic diesel fuel yield increase are based on typical yield responses for increasing the TBP cut point between the SPK or synthetic diesel and the recycle oil from the current state-of-the-art process cut point to the cut points in the present invention.

The molecular sieves can optionally be arranged in a swing bed arrangement. One bed can be fed with the heavy distillate stream 150 or the reactor effluent from an isomerization and hydrocracking zone stream 225 from a synthetic distillate fuel process with recycle. Once the adsorbing bed reaches its capacity of n-paraffin adsorption, such that any further breakthrough of n-paraffins would not achieve the desired cold flow property requirement, the bed can be taken off-line and regenerated with a desorbent to desorb the n-paraffins. The desorbed mixture can then be separated in a fractionation column into a desorbent stream and a stream 165 containing n-paraffins from heavy distillate stream or a stream 240 containing n-paraffins from the isomerization and cracking zone. The desorbent is reused, and stream 165 or 240 is recycled back to the reactor for isomerization and cracking. The regenerated adsorbent bed can then be placed back in service in an adsorbing function. The system can be designed so that two or more beds are used. One bed can be adsorbing, and the other bed or beds can be being desorbed and/or prepared to be placed back in service. Suitable desorbents include, but are not limited to, light n-paraffins such as propane, n-butane, n-pentane, mixtures of light n-paraffins, and the like.

In a process such as is shown in FIG. 2, the amount of n-paraffins being adsorbed would be much lower than in a process such as FIG. 3, where the whole reactor effluent stream from the isomerization and hydrocracking zone is sent over the molecular sieves. In the embodiment shown in FIG. 2, the beds of molecular sieves could be much smaller.

### EXAMPLE

Aviation fuel and arctic diesel were produced using the current invention. A biorenewable feedstock was hydrotreated in a hydrotreating zone under hydrotreatment conditions to produce a mixture comprising almost entirely n-paraffins. The mixture from the hydrotreatment zone was processed in an isomerization and hydrocracking zone using mild conditions as described above to produce a synthetic diesel, or a “Green Diesel”, with the properties shown in Table 1 below. The isomer to n-paraffin ratio in the synthetic diesel carbon number range from the isomerization and hydrocracking zone was 4:1. In the first example, the green diesel was passed through a bed of molecular sieve to completely remove the n-paraffin and produce an aviation fuel that meets the flash point and freeze point specifications of Jet A or Jet A-1. The n-paraffins in the green diesel were retained in the bed of molecular sieve. In the second example, the green diesel was passed through a bed of molecular sieve to substantially remove the n-paraffin. A small portion of the n-paraffins from the green diesel feed were allowed to breakthrough into Product 2 such that the n-paraffin concentration is 2.4 wt %. Product 2 meets flash point and cold flow properties specifications for an arctic diesel. The n-paraffins in the green diesel were substantially retained in the bed of molecular sieve.

TABLE 1

| | Green | Product 1 After | Product 2 After | |
|---|---|---|---|---|
| | Diesel | Molecular | Molecular | |
| | Feed to | Sieve | Sieve | Specs |
| | Molecular | Adsorption of | Adsorption of | for JetA |
| | Sieve Bed | n-paraffins | n-paraffins | (Jet-A1) |
| Freeze Point, | −1.1 | −47.5 | −37.7 | −40 (−47) |
| ° C. | | | | |
| n-paraffins, % | 20.0 | 0 | 2.4 | Not |
| | | | | applicable |
| Density | .7791 | .7827 | .7805 | .775-.840 |
| (15° C./15° C.) | | | | |
| Iso:Normal | 3.99 | Infinite | 40 | Not |
| Ratio | | | | applicable |
| Flash Point, | 68 | 68 | 68 | 38 min |
| ° C. | | (estimated) | (estimated) | |

The term biorenewable feedstock is meant to include feedstocks other than those obtained directly from petroleum crude oil. Another term that has been used to describe this class of feedstocks is biorenewable fats and oils. The biorenewable feedstocks that can be used in the present invention include any of those which comprise glycerides and free fatty acids (FFA). Examples of these feedstocks include, but are not limited to, canola oil, corn oil, soy oils, rapeseed oil, soybean oil, colza oil, tall oil, sunflower oil, hempseed oil, olive oil, linseed oil, coconut oil, castor oil, peanut oil, palm oil, mustard oil, cottonseed oil, tallow, yellow and brown greases, lard, train oil, fats in milk, fish oil, algal oil, sewage sludge, cuphea oil, camelina oil, jatropha oil, curcas oil, babassu oil, palm kernel oil, crambe oil, and the like. Biorenewable is another term used to describe these feedstocks. The glycerides, FFAs, and fatty acid alkyl esters, of the typical vegetable oil or animal fat contain aliphatic hydrocarbon chains in their structure which have about 8 to about 24 carbon atoms with a majority of the oils containing high concentrations of fatty acids with 16 and 18 carbon atoms. Mixtures or co-feeds of renewable feedstocks and fossil fuel derived hydrocarbons may also be used as the feedstock. Other feedstock components may be used if the carbon chain length is well-defined before mixing with renewable oils to allow meeting desired yields and specifications for diesel and aviation range paraffins.

Various additives may be combined with the aviation fuel composition generated in order to meet required specifications for different specific fuels. The specifications could include physical characteristics, chemical characteristics, or both. The specifications could be industry standard, government, and/or military fuel standard specifications. In particular, the hydrocarbon product stream in the aviation fuel range generated herein complies with, is a blending component for, or may be combined with one or more additives to meet at least one of: ASTM D 7566 Specification for Aviation Turbine Fuel Containing Synthesized Hydrocarbons, ASTM D 1655 Specification for Aviation Turbine Fuels Defense Stan 91-91 Turbine Fuel, Aviation Kerosene Type, Jet A-1 NATO code F-35, F-34, F-37 Aviation Fuel Quality Requirements for Jointly Operated Systems (Joint Checklist) A combination of ASTM and Def Stan requirements GOST 10227 Jet Fuel Specifications (Russia) Canadian CAN/CGSB-3.22 Aviation Turbine Fuel, Wide Cut Type Canadian CAN/CGSB-3.23 Aviation Turbine Fuel, Kerosene Type MIL-DTL-83133, JP-8, MW-DTL-5624, JP-4, JP-5 QAV-1 (Brazil) Especifcacao de Querosene de Aviacao No. 3 Jet Fuel (Chinese) according to GB6537 DCSEA 134A (France) Carbureacteur Pour Turbomachines D'Aviation, Type Kerosene Aviation Turbine Fuels of other countries, meeting the general grade requirements for Jet A, Jet A-1, Jet B, and TS-1 fuels as described in the IATA Guidance Material for Aviation Turbine Fuel Specifications. Additives may be added to the jet fuel in order to meet particular specifications. One particular type of jet fuel is JP-8, defined by Military Specification ML-DTL-83133, which is a military grade type of highly refined kerosene based jet propellant specified by the United States Government.

The feedstocks used in the present invention may contain a variety of impurities. For example, tall oil is a byproduct of the wood processing industry, and it contains esters and rosin acids in addition to FFAs. Rosin acids are cyclic carboxylic acids. The renewable feedstocks may also contain contaminants such as alkali metals, e.g. sodium and potassium, phosphorous, as well as solids, water and detergents. An optional first step is to remove as much of these contaminants as possible. Any known pretreatment steps can be used including, but not limited to, contacting the renewable feedstock with an ion-exchange resin in a pretreatment zone at pretreatment conditions, contacting the renewable feedstock with a bleaching earth, such as bentonite clay, in a pretreatment zone, mild acid washing, the use of guard beds, filtration and solvent extraction techniques, hydroprocessing, such as that described in U.S. application Ser. No. 11/770,826, hydrolysis may be used to convert triglycerides to a contaminant mixture of free fatty acids, and hydrothermolysis may be used to convert triglycerides to oxygenated cycloparaffins, or combinations thereof.

The feedstocks are flowed to the hydroprocessing zone or stage comprising one or more catalyst beds in one or more reactor vessels. The invention comprises two hydroprocessing zones—a hydrotreatment zone and an isomerization and hydrocracking zone. Within the hydroprocessing reaction zone or stage, multiple beds or vessels may be employed, and where multiple beds or vessels are employed, interstage product separation may or may not be performed between the beds or vessels. The term feedstock is meant to include feedstocks that have not been treated to remove contaminants, as well as those feedstocks purified in a pretreatment zone or an oil processing facility. The feedstocks, with or without additional liquid recycled from one or more product streams, may be mixed in a feed tank upstream of the reaction zone, mixed in the feed line to the reactor, or mixed in the reactor itself. In the reaction zone, the feedstocks are contacted with a multifunctional catalyst or set of catalysts that perform deoxygenation, hydrogenation, desulfurization, denitrification, and isomerization functions in the presence of hydrogen.

A number of reactions occur concurrently within the hydrotreatment zone. The order of the reactions is not critical to the invention, and the reactions may occur in various orders. One reaction occurring in the reaction zone is hydrogenation to saturate olefinic compounds in the reaction mixture. Another type of reaction occurring in the reaction zone is deoxygenation. The deoxygenation of the mixture may proceed through different routes such as decarboxylation, where the feedstock oxygen is removed as carbon dioxide, decarbonylation, where the feedstock oxygen is removed as carbon monoxide, and/or hydrodeoxygenation, where the feedstock oxygen is removed as water. Decarboxylation, decarbonylation, and hydrodeoxygenation are herein collectively referred to as deoxygenation reactions.

Sufficient isomerization to prevent poor cold flow properties is needed. Aviation fuel and aviation blending components must have better cold flow properties than is achievable with essentially all n-paraffins. At least a portion of the n-paraffins are isomerized to branched paraffins in the isomerization and hydrocracking zone. The extent of isomerization needed is dependent on the value of the cold flow property specifications required for the final fuel product. Some fuels require a lower cloud or freeze point, and thus need a greater extent of reaction from the isomerization reaction to produce a larger concentration of branched-paraffins. The required cold flow property requirements can also be achieved by hydrocracking to reduce the average carbon number of the products relative to the feedstock while also producing isomers of lower average carbon number relative to the feedstock. The method of the invention involves minimizing hydrocracking and maximizing isomerization to achieve the desired cold flow property values in the products. The catalyst function for deoxygenation and hydrogenation will be similar to those already known for hydrogenation or hydrotreating. The deoxygenation and hydrogenation functions, which may be the same or separate active sites, may be noble metals such as a platinum group metals including but not limited to ruthenium, rhodium, palladium, platinum, and mixtures thereof, supported on a high surface area carrier material such as alumina, silica, silica-alumina, magnesium oxide, titania, zirconia, activated carbon and others known in the art, at levels ranging from about 0.05 to about 10 weight-% of the catalytic composite. Examples of other active sites that may be employed to provide the deoxygenation and hydrogenation functions are sulfided base metals such as sulfided NiMo or sulfided NiW or a sulfided CoMo. A base metal is a metal which oxidizes when heated in air, and other base metals, in addition to nickel, molybdenum and tungsten, which may be a catalyst component herein include iron, lead, zinc, copper, tin, germanium, chromium, titanium, rhenium, indium, gallium, uranium, dysprosium, thallium and mixtures and compounds thereof. Sulfided base metal catalysts may optionally be supported on carrier material such as alumina, silica, silica-alumina, magnesium oxide, activated carbon and others known in the art, or may alternately be used without additional support components,

Catalyst functions and conditions for isomerization are well known in the art. See for example US 2004/0230085 A1 which is incorporated by reference in its entirety. Due to the presence of hydrogen, these reactions may also be called hydroisomerization.

Overall, the isomerization and hydrocracking of the paraffinic product can be accomplished in any manner known in the art or by using any suitable catalyst known in the art. In general, catalysts or catalytic components having an acid function and mild hydrogenation function are favorable for catalyzing the isomerization and hydrocracking reactions. For a single multi-component catalyst, the same active site employed for deoxygenation can also serve as the mild hydrogenation function for the isomerization reactions. In general, suitable isomerization and hydrocracking catalysts comprise a metal of Group VIII (IUPAC 8-10) of the Periodic Table and a support material. Suitable Group VIII metals include platinum and palladium, each of which may be used alone or in combination. (Any mention of base metals here? to be inclusive of hydrocracking catalysts) The support material may be amorphous or crystalline, or a combination of the two. Suitable support materials include, aluminas, amorphous aluminas, amorphous silica-aluminas, ferrierite, ALPO-31, SAPO-11, SAPO-31, SAPO-37, SAPO-41, SM-3, MgAPSO-31, FU-9, NU-10, NU-23, ZSM-12, ZSM-22, ZSM-23, ZSM-35, ZSM-48, ZSM-50, ZSM-57, MeAPO-11, MeAPO-31, MeAPO-41, MeAPSO-11, MeAPSO-31, MeAPSO-41, MeAPSO-46, ELAPO-11, ELAPO-31, ELAPO-41, ELAPSO-11, ELAPSO-31, ELAPSO-41, laumontite, cancrinite, offretite, hydrogen form of stillbite, magnesium or calcium form of mordenite, and magnesium or calcium form of partheite, each of which may be used alone or in combination. ALPO-31 is described in U.S. Pat. No. 4,310,440. SAPO-11, SAPO-31, SAPO-37, and SAPO-41 are described in U.S. Pat. No. 4,440,871. SM-3 is described in U.S. Pat. Nos. 4,943,424; 5,087,347; 5,158,665; and U.S. Pat. No. 5,208,005. MgAPSO is a MeAPSO, which is an acronym for a metal aluminumsilicophosphate molecular sieve, where the metal Me is magnesium (Mg). Suitable MeAPSO-31 catalysts include MgAPSO-31. MeAPSOs are described in U.S. Pat. No. 4,793,984, and MgAPSOs are described in U.S. Pat. No. 4,758,419. MgAPSO-31 is a preferred MgAPSO, where 31 means a MgAPSO having structure type 31. Many natural zeolites, such as ferrierite, that have an initially reduced pore size can be converted to forms suitable for isomerization by removing associated alkali metal or alkaline earth metal by ammonium ion exchange and calcination to produce the substantially hydrogen form, as taught in U.S. Pat. No. 4,795,623 and U.S. Pat. No. 4,924,027. Further catalysts and conditions for skeletal isomerization are disclosed in U.S. Pat. Nos. 5,510,306, 5,082,956, and U.S. Pat. No. 5,741,759.

The isomerization catalyst function may also comprise a modifier selected from the group consisting of lanthanum, cerium, praseodymium, neodymium, phosphorus, samarium, gadolinium, terbium, and mixtures thereof, as described in U.S. Pat. No. 5,716,897 and U.S. Pat. No. 5,851,949. Other suitable support materials include ZSM-22, ZSM-23, and ZSM-35, which are described for use in dewaxing in U.S. Pat. No. 5,246,566 and in the article entitled “New molecular sieve process for lube dewaxing by wax isomerization,” written by S. J. Miller, in Microporous Materials 2 (1994) 439-449. The teachings of U.S. Pat. Nos. 4,310,440; 4,440,871; 4,793,984; 4,758,419; 4,943,424; 5,087,347; 5,158,665; 5,208,005; 5,246,566; 5,716,897; and U.S. Pat. No. 5,851,949 are hereby incorporated by reference.

U.S. Pat. No. 5,444,032 and U.S. Pat. No. 5,608,968 teach a suitable bifunctional catalyst which is constituted by an amorphous silica-alumina gel and one or more metals belonging to Group VIIIA, and is effective in the hydroisomerization of long-chain normal paraffins containing more than 15 carbon atoms. U.S. Pat. Nos. 5,981,419 and 5,908,134 teach a suitable bifunctional catalyst which comprises: (a) a porous crystalline material isostructural with beta-zeolite selected from boro-silicate (BOR—B) and boro-alumino-silicate (Al—BOR—B) in which the molar SiO₂:Al₂O₃ ratio is higher than 300:1; (b) one or more metal(s) belonging to Group VIIIA, selected from platinum and palladium, in an amount comprised within the range of from 0.05 to 5% by weight. Article V. Calemma et al., App. Catal. A: Gen., 190 (2000), 207 teaches yet another suitable catalyst.

The isomerization and hydrocracking zone may contain one or more beds of the same catalysts or of different fixed catalysts, which respond to modification in conversion and/or reaction characteristics of the final product with ammonia changes. In one aspect, when the desired products are diesel, or middle distillates, suitable hydrocracking catalysts utilize amorphous bases or low-level zeolite bases or both combined with one or more Group VIII or Group VIB metal hydrogenating components. In another aspect, when the desired products are in the gasoline, or naphtha boiling ranges, the isomerization and hydrocracking zone contains a catalyst which comprises, in general, any amorphous bases or crystalline zeolite cracking base or both upon which is deposited a proportion of a Group VIII or Group VIB metal hydrogenating component.

Additional hydrogenating components may be selected from Group VIB for incorporation with the zeolite base. The zeolite cracking bases are sometimes referred to in the art as molecular sieves and are usually composed of silica, alumina and one or more exchangeable cations such as sodium, magnesium, calcium, rare earth metals, and the like. They are further characterized by crystal pores of relatively uniform diameter between about 4 and 14 Angstroms (10⁻¹⁰ meters). It is preferred to employ zeolites having a relatively high silica/alumina mole ratio between about 3 and 25. Suitable zeolites found in nature include, for example, mordenite, stilbite, heulandite, ferrierite, dachiardite, chabazite, erionite and faujasite. Suitable synthetic zeolites include, for example, the B, X, Y, beta and L crystal types, e.g., synthetic faujasite and mordenite. The preferred zeolites are those having crystal pore diameters between about 7 and 12 Angstroms (10⁻¹⁰ meters), wherein the silica/alumina mole ratio is about 4 to 12. One example of a zeolite falling in the preferred group is synthetic Y molecular sieve.

The natural occurring zeolites are normally found in a sodium form, an alkaline earth metal form, or mixed forms. The synthetic zeolites are nearly always prepared first in the sodium form. In any case, for use as a cracking base it is preferred that most or all of the original zeolitic monovalent metals be ion-exchanged with a polyvalent metal and/or with an ammonium salt followed by heating to decompose the ammonium ions associated with the zeolite, leaving in their place hydrogen ions and/or exchange sites which have actually been decationized by further removal of water. Hydrogen or “decationized” Y zeolites of this nature are more particularly described in U.S. Pat. No. 3,130,006.

Mixed polyvalent metal-hydrogen zeolites may be prepared by ion-exchanging first with an ammonium salt, then partially back exchanging with a polyvalent metal salt and then calcining. In some cases, as in the case of synthetic mordenite, the hydrogen forms can be prepared by direct acid treatment of the alkali metal zeolites. The preferred cracking bases are those which are at least about 10 percent, and preferably at least 20 percent, metal-cation-deficient, based on the initial ion-exchange capacity. A specifically desirable and stable class of zeolites are those wherein at least about 20 percent of the ion exchange capacity is satisfied by hydrogen ions.

The active metals employed in the preferred hydrocracking catalysts of the present invention as hydrogenation components are those of Group VIII, i.e., iron, cobalt, nickel, ruthenium, rhodium, palladium, osmium, iridium and platinum. In addition to these metals, other promoters may also be employed in conjunction therewith, including the metals of Group VIB, e.g., molybdenum and tungsten. The amount of hydrogenating metal in the catalyst can vary within wide ranges. Broadly speaking, any amount between about 0.05 and 30 wt-% may be used. In the case of the noble metals, it is normally preferred to use about 0.05 to about 2 wt-%. The preferred method for incorporating the hydrogenating metal is to contact the zeolite base material with an aqueous solution of a suitable compound of the desired metal wherein the metal is present in a cationic form. Following addition of the selected hydrogenating metal or metals, the resulting catalyst powder is then filtered, dried, pelleted with added lubricants, binders or the like if desired, and calcined in air at temperatures of, e.g., about 700.degree. to about 1200.degree. F. (about 371.degree. to about 648.degree. C.) in order to activate the catalyst. Alternatively, the catalyst base component may first be pelleted, calcined and followed by the addition of the hydrogenating component and oxidation of the metals on the catalyst base.

The foregoing catalysts may be employed in undiluted form, or the powdered zeolite catalyst may be mixed and co-pelleted with other relatively less active catalysts, diluents or binders such as alumina, silica gel, silica-alumina cogels, activated clays and the like in proportions ranging between 5 and 90 wt-%. These diluents may be employed as such or they may contain a minor proportion of an added hydrogenating metal such as a Group VIB and/or Group VIII metal. Additional metal promoted hydrocracking catalysts may also be utilized in the process of the present invention which comprises, for example, aluminophosphate molecular sieves, crystalline chromosilicates and other crystalline silicates.

Selection of the hydrocracking catalysts and operating parameters influences the catalyst activity, efficiency and selectivity and therefore product output from the isomerization and hydrocracking zone, in terms of the mix of hydrocarbon constituents of the output stream, (e.g., the hydrocarbon chain length distribution, the alkane and naphtha content, etc.).

The isomerization and hydrocracking zone can contain one or more hydroisomerization or hydrocracking catalysts or a catalyst or catalysts that perform both hydroisomerization and hydrocracking functions.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A method of making synthetic distillate fuel comprising:
hydrotreating a feedstock in a hydrotreating zone under hydrotreating conditions to obtain a mixture of substantially n-paraffins;
isomerizing and hydrocracking at least a portion of the n-paraffins in an isomerization and hydrocracking zone under mild isomerization and hydrocracking conditions to obtain a mixture of n-paraffins and isomerized paraffins;
separating at least a portion of the mixture of n-paraffins and isomerized paraffins in a bed of molecular sieves into an n-paraffin stream consisting essentially of n-paraffins and an isomerized paraffin stream consisting essentially of isomerized paraffins;
fractionating the isomerized paraffin stream into at least a heavy distillate fraction consisting essentially of isomerized paraffins, and a light distillate fraction consisting essentially of isomerized paraffins.

2. The method of claim 1 further comprising recycling the n-paraffin stream to the isomerization and hydrocracking zone.

3. The method of claim 2 further comprising recycling at least a portion of the heavy distillate fraction to the isomerization and hydrocracking zone.

4. The method of claim 1 wherein the mild isomerization and hydrocracking conditions comprise a temperature in a range of about 260 to about 345° C. (500 to 650° F.), a pressure of about 1750 kPa(g) (about 250 psig) to about 6900 kPa(g) (about 1000 psig), and a ratio of H₂:HC of about 1000 to about 5000 standard cubic feet per barrel (SCFB), a LHSV of about 0.1 to 5, producing a mixture with a ratio of isomerized paraffins to n-paraffins of less than 7:1.

5. The method of claim 1 wherein separating at least a portion of the mixture of n-paraffins and isomerized paraffins in a bed of molecular sieves comprises adsorbing the n-paraffins in the bed of molecular sieves.

6. The method of claim 5 further comprising desorbing the adsorbed n-paraffins from the bed of molecular sieves using a desorbent forming a desorbent mixture of n-paraffins and desorbent.

7. The method of claim 6 further comprising fractionating the desorbent mixture into a desorbent stream and an n-paraffin stream.

8. The method of claim 1 wherein there are at least two beds of molecular sieves.

9. The method of claim 1 wherein the feedstock comprises biorenewable feedstocks, biorenewable Fischer-Tropsch liquids, and non-biorenewable Fischer-Tropsch liquids.

10. The method of claim 1 wherein the true boiling point cut point between the light distillate fraction and the recycled n-paraffin stream and portion of the heavy distillate fraction is at least about 287° C.

11. A method of making synthetic distillate fuel comprising:
hydrotreating a feedstock in a hydrotreating zone under hydrotreating conditions to obtain a mixture of substantially n-paraffins;
isomerizing and hydrocracking at least a portion of the n-paraffins in an isomerization and hydrocracking zone under mild isomerization and hydrocracking conditions to obtain a mixture of n-paraffins and isomerized paraffins;
fractionating the mixture of n-paraffins and isomerized paraffins into at least a heavy distillate fraction, and a light distillate fraction;
separating the heavy distillate fraction in a bed of molecular sieves into a heavy distillate n-paraffin stream consisting essentially of n-paraffins and a heavy distillate isomerized paraffin stream consisting essentially of isomerized paraffins.

12. The method of claim 11 further comprising recycling at least a portion of the heavy distillate n-paraffin stream to the isomerization and hydrocracking zone.

13. The method of claim 12 further comprising combining the heavy distillate isomerized paraffin stream with the light distillate fraction.

14. The method of claim 11 wherein the mild isomerization and hydrocracking conditions comprise a temperature in a range of about 260 to about 345° C. (500 to 650° F.), a pressure of about 1750 kPa(g) (about 250 psig) to about 6900 kPa(g) (about 1000 psig), and a ratio of H₂:HC of about 1000 to about 5000 standard cubic feet per barrel (SCFB), a LHSV of about 0.1 to 5, producing a mixture with a ratio of isomerized paraffins to n-paraffins of less than 7:1.

15. The method of claim 11 wherein separating at least a portion of the mixture of n-paraffins and isomerized paraffins in a bed of molecular sieves comprises adsorbing the n-paraffins in the bed of molecular sieves.

16. The method of claim 15 further comprising desorbing the adsorbed n-paraffins from the bed of molecular sieves using a desorbent forming a desorbent mixture of n-paraffins and desorbent.

17. The method of claim 16 further comprising fractionating the desorbent mixture into a desorbent stream and an n-paraffin stream.

18. The method of claim 11 wherein there are at least two beds of molecular sieves.

19. The method of claim 11 wherein the feedstock comprises biorenewable feedstocks, biorenewable Fischer-Tropsch liquids, and non-biorenewable Fischer-Tropsch liquids.

20. The method of claim 13 wherein the true boiling point cut point between the combination of the light distillate fraction and the heavy distillate isomerized paraffin stream and the recycled heavy distillate n-paraffins stream is at least about 287° C.
